(19) United States
(12) Patent Application Publication
(10) Pub. No.: US 2022/0243190 A1
(43) Pub. Date: Aug. 4, 2022

(54) 3D PRINTING OF BIOFILMS

(71) Applicant: University of Rochester, Rochester, NY (US)

(72) Inventor: Anne S. Meyer, Fairport, NY (US)

(21) Appl. No.: 17/626,532

(22) PCT Filed: Jul. 13, 2020
(86) PCT No.: PCT/US20/41805

§ 371 Date: Jan. 12, 2022

Related U.S. Application Data
(60) Provisional application No. 62/873,649, filed on Jul. 12, 2019.

(51) Int. Cl. C12N 11/04; C12N 1/20; B33Y 10/00; B33Y 30/00; B33Y 80/00

(52) U.S. Cl. CPC C12N 11/04; C12N 1/20; B33Y 80/00; B33Y 30/00; B33Y 10/00

(57) ABSTRACT

The present disclosure provides easy and cost-effective methods for 3D printing of microorganisms to form biofilms, such as genetically engineered Escherichia coli biofilms. In some embodiments, the 3D printing platform exploits simple alginate chemistry for printing of a bacteria-alginate bioink mixture onto calcium-containing agar surfaces, resulting in the formation of bacteria-encapsulating hydrogels with varying geometries. Bacteria in these hydrogels remain intact, spatially patterned, and viable for several days. Printing of engineered bacteria to produce inducible biofilms leads to formation of multilayered three-dimensional structures that can tolerate harsh chemical treatments, enabling the construction of living biofilm-derived materials in a large-scale and environmentally-stable manner.

### CROSS-REFERENCE TO RELATED APPLICATIONS

This application claims priority to U.S. Provisional Patent Application No. 62/873,649, filed Jul. 12, 2019, the contents of which are incorporated by reference herein in their entirety.

### STATEMENT REGARDING FEDERALLY SPONSORED RESEARCH OR DEVELOPMENT

This invention was made with government support under FA2386-18-1-4059 awarded by The Air Force Research Laboratory. The government has certain rights in the invention.

### BACKGROUND

Bacterial biofilms are organic platforms for sustainable nano- or biomaterials production and processing. The matrix components of naturally-occurring biofilms are resilient to extreme conditions and demonstrate self-assembly and spatial patterning (Bjarnsholt T et al., J Intern Med, 2018, 284:332-345; Flemming H C et al., Nat Rev Microbiol, 2016, 14:563-575; Felz S et al., Water Res, 2019, 157:201-208). These features explain why biofilms have recently become hotspots in emerging materials fabrication and additive manufacturing technologies. Biofilm-derived materials have been applied to a diverse range of applications from detoxification of chemicals to personalized human medicine. By using tools of synthetic biology, it is now possible to improve existing functionalities or even add new functions to biofilm-forming bacteria. Such engineered biofilms are constructed by creating genetic fusions in which desired heterologous functional peptides are appended onto biofilm matrix proteins. These chimeric proteins are then actively secreted by the engineered bacteria and self-assemble in the extracellular matrix of the biofilms (Nguyen P Q et al., Nat Commun, 2014, 5:4945; Chen A Y et al., Nat Mater, 2014, 13:515-523). Synthetic biofilms can exhibit new functionalities deriving from the added peptides while simultaneously retaining their natural functionalities such as resilience, long-term viability, and self-regeneration (Huang J et al., Nat Chem Biol, 2019, 15:34-41). Genetically tractable bacteria such as Escherichia coli and Bacillus subtilis have been successfully employed for the creation of synthetic biofilms and engineered materials (Nguyen P Q et al., Nat Commun, 2014, 5:4945; Huang J et al., Nat Chem Biol, 2019, 15:34-41). During the creation of synthetic biofilms, various factors must be evaluated, including the determination of optimal peptide fusion sites, the tolerance of the fusion protein to mutations, the toxicity of the new peptide tags to the bacterial cells, and appropriate functional assays for characterization of the novel biofilm functionalities. The resultant biofilm-derived materials can exhibit marked advantages over materials fabricated by planktonic bacteria cultures, in terms of their resistance to extreme and unexpected environments, reusability, spatial multi-scale patterning, and tunable properties.

Thus, there is a need in the art for improved biofilms and methods of making the same. The present disclosure satisfies this need.

### SUMMARY

In one aspect, the present disclosure provides a biofilm, comprising: a layer of matrix material; and at least one population of microorganisms positioned within the layer of matrix material; wherein the at least one population of microorganisms produce at least one molecule that permeates the matrix material.

In one embodiment, the at least one population of microorganisms is selected from the group consisting of: bacteria, viruses, cells, protozoa, amoeba, algae, and fungi. In one embodiment, the at least one population of microorganisms is genetically altered. In one embodiment, the at least one population of microorganisms is selected from the group consisting of: Escherichia coli, Bacillus subtilis, Bacteroides fragilis, Bifidobacterium bifidum, Enterobacter cloacae, Enterococcus faecalis, Methanobrevibacter smithii, Neisseria meningitides, Neisseria sicca, Staphylococcus aureus, Staphylococcus epidermidis, Streptococcus mutans, Streptococcus sanguinis, Streptococcus gordonii, Streptococcus salivarius, and Actinomyces naeslundii. In one embodiment, the layer of matrix material further comprises one or more populations of host cells.

In one embodiment, the layer of matrix material is selected from the group consisting of: gelatin, agarose, hyaluronic acid, fumed silica, κ-carrageenan, cellulose, collagen, fibrin, fibrinogen, thrombin, elastin, laminin, fibronectin, vitronectin, chondroitin 4-sulfate, chondroitin 6-sulfate, dermatan sulfate, heparin sulfate, vixapatin (VP12), heparin, and keratan sulfate, proteoglycans, chitin, chitosan, alginic acids, alginates, and combinations thereof. In one embodiment, the layer of matrix material further comprises one or more matrix molecule selected from the group consisting of: proteins, peptides, enzymes, amino acids, nucleic acids, vitamins, hormones, antibodies, growth factors, nanoparticles, microparticles, liposomes, viral and non-viral transfection systems, therapeutics, and drugs.

In one embodiment, the biofilm is fabricated by the deposition of a bioink on a substrate, the bioink comprising the at least one population of microorganisms and a matrix material. In one embodiment, the substrate is selected from the group consisting of: gelatin, agarose, hyaluronic acid, fumed silica, κ-carrageenan, cellulose, collagen, fibrin, fibrinogen, thrombin, elastin, laminin, fibronectin, vitronectin, chondroitin 4-sulfate, chondroitin 6-sulfate, dermatan sulfate, heparin sulfate, vixapatin (VP12), heparin, and keratan sulfate, proteoglycans, chitin, chitosan, alginic acids, alginates, metals, glass, wood, fabrics, fibers, polymers, plastics, and combinations thereof.

In one embodiment, the fabrication of the biofilm further includes the application of a polymerizer or a crosslinker. In one embodiment, the polymerizer or crosslinker comprises calcium. In one embodiment, the deposition is a method selected from the group consisting of: 3D printing, inkjet printing, extrusion, screen printing, electrospinning, spin coating, sputtering, rolling, and spraying.

In one embodiment, the biofilm comprises enzymes suitable for processing materials under extreme pH, temperature, and solvent conditions. In one embodiment, the biofilm is an anti-fouling coating having bacteria that produce anti-corrosive compounds. In one embodiment, the biofilm is a detoxifying matrix having catabolic enzymes, heavy metal binding proteins, inorganic nanoparticles, rare earth element (REE) binding domains, and combinations thereof. In one embodiment, the biofilm is recyclable and reusable.

In one embodiment, the biofilm further comprises one or more additional layers of matrix material. In one embodiment, at least one of the additional layers of matrix material comprises at least one population of microorganisms. In one embodiment, the additional layers of matrix material each comprise different populations of microorganisms.

In one aspect, the present disclosure provides a method of fabricating a biofilm, comprising the steps of: providing at least one bioink, each bioink comprising at least one population of microorganisms suspended in a matrix material; and depositing the at least one bioink onto a substrate; wherein the at least one population of microorganisms produce at least one molecule that permeates the matrix material.

In one embodiment, the substrate is a suspension media comprising a non-Newtonian fluid, such that the at least one bioink is depositable in three-dimensional space within the suspension media

In one embodiment, the at least one bioink is extruded through a passive mixer with at least one additional composition, wherein the passive mixer comprises at least two inlet channels fluidly joining together into a mixing channel that is fluidly connected to an outlet channel. In one embodiment, the mixing channel comprises at least one turbulence-increasing physical structure. In one embodiment, the physical structure is selected from the group consisting of: channel path undulations, embedded pegs, embedded nodules, and embedded fins.

In one embodiment, the at least one bioink is extruded through a multi-input printing nozzle with at least one additional composition, wherein the multi-input nozzle comprises at least one first nozzle having a first nozzle tip and at least one second having a second nozzle tip. In one embodiment, the first nozzle tip has a diameter that is smaller than a diameter of the second nozzle tip. In one embodiment, the first nozzle tip is nested within the diameter of the second nozzle tip. In one embodiment, the first nozzle tip is positioned adjacent to the second nozzle tip. In one embodiment, the first nozzle tip and the second nozzle tip terminate at an equal position relative to the multi-input printing nozzle. In one embodiment, the first nozzle tip and the second nozzle tip terminate at different positions relative to the multi-input printing nozzle.

### BRIEF DESCRIPTION OF THE DRAWINGS

The following detailed description of exemplary embodiments will be better understood when read in conjunction with the appended drawings. It should be understood, however, that contemplated embodiments are not limited to the precise arrangements and instrumentalities shown in the drawings.

FIG. 1 is a schematic depicting a bioink deposited on a substrate to form a biofilm with encapsulated microorganisms that naturally produce a complex network of proteins and peptides over time.

FIG. 2 is a schematic depicting applications of 3D-printed synthetic biofilms. Bacteria can be genetically engineered to produce structural biofilm proteins (in blue) decorated with specific functional peptides (in green) via heterologous expression in a bacterial strain that has a genetic deletion for structural biofilm proteins. By combining these engineered bacteria with 3D bioprinting, 3D-printed engineered biofilms can be created with multiple potential applications, including (A) Environmental detoxification and bioremediation, (B) Biomedical applications, (C) Tunable materials production with improved mechanical and/or conductive properties, (D) Fabrication of responsive materials, (E) Biocatalysis-driven materials processing, (F) Addressing fundamental research questions, and (G) Creation of reproducible model biofilm systems for studying the structure-function relationships of bacterial biofilm.

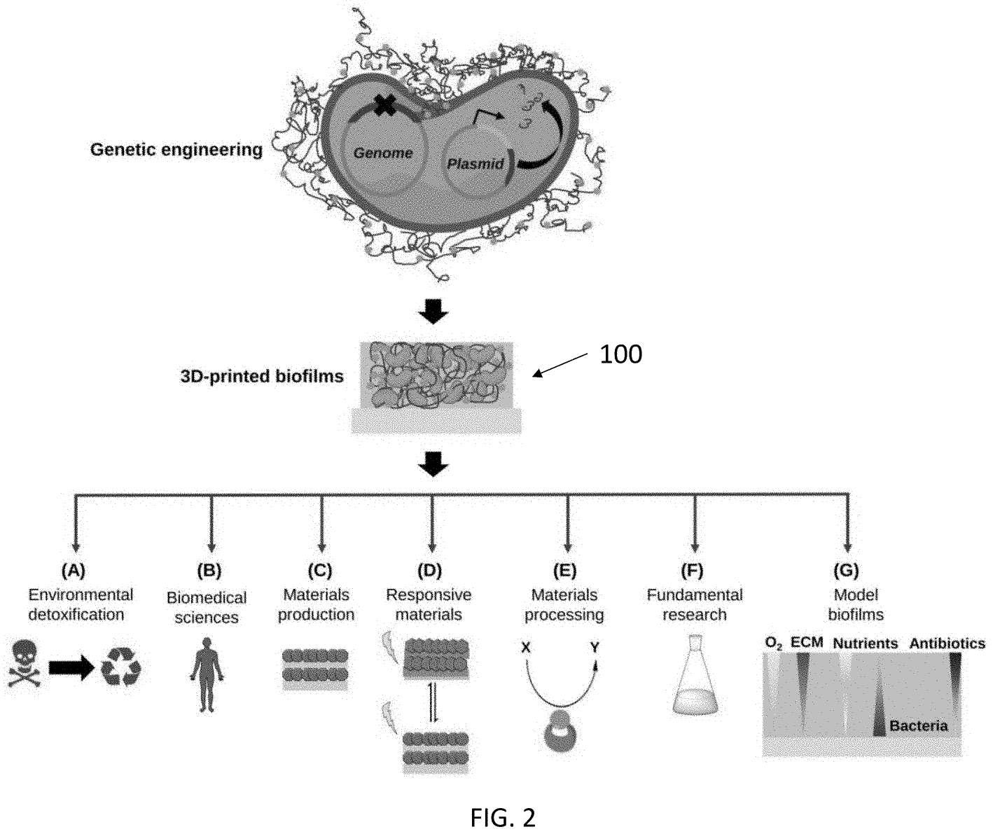

FIG. 3A and FIG. 3B depict the results of experiments demonstrating free-form printing of arbitrary shapes. FIG. 3A depicts an exemplary process of 3D bacteria printing within an agarose slurry. FIG. 3B depicts the achievement of a 3D-printed hydrogel structure with aspect ratios and internal cavities that cannot be achieved via printing in air.

FIG. 4A through FIG. 4C depict an exemplary passive mixer for multichannel printing. FIG. 4A is a side view of a transparent polymer block encapsulating a Y-shaped series of channels combining two sources of ink into one output channel. FIG. 4B depicts the results of input inks exhibiting low initial mixing due to low Reynolds number. FIG. 4C depicts the results of passage through a microfluidics channel lined with semicircular winding that introduces turbulent flow, ensuring mixing of multichannel inputs.

FIG. 5 depicts a multi-input printing nozzle (left) and a magnified view of the nozzle tip (right). The nozzle is created by threading a narrow-gauge syringe needle through a wider gauge syringe needle, resulting in an extrudable solution of crosslinking agents (output through the outer diameter syringe needle) that encapsulates ink (output through the inner diameter syringe needle).

FIG. 6 depicts the results of experiments quantifying colony forming units of 3D-printing biofilms containing E. coli Nissle, K-12 E. coli containing an empty plasmid (Curli−), or K-12 E. coli containing a plasmid expressing CsgA curli fibers (Curli+), exposed to no treatment (control), 30%, 50%, or 70% ethanol for 5 minutes.

FIG. 7 depicts the results of experiments quantifying soluble oxygen concentrations at a range of depths within agar or within 3D-printed structures printed on top of agar, where the 3D-printed bio-ink containing no bacteria, K-12 E. coli containing an empty plasmid (Curli−), or K-12 E. coli containing a plasmid expressing CsgA curli fibers (Curli+).

### DETAILED DESCRIPTION

The present disclosure provides easy and cost-effective methods for 3D printing of microorganisms to form biofilms, such as genetically engineered Escherichia coli biofilms. In some embodiments, the 3D printing platform exploits simple alginate chemistry for printing of a bacteria-alginate bioink mixture onto calcium-containing agar surfaces, resulting in the formation of bacteria-encapsulating hydrogels with varying geometries. Bacteria in these hydrogels remain intact, spatially patterned, and viable for several days. Printing of engineered bacteria to produce inducible biofilms leads to formation of multilayered three-dimensional structures that can tolerate harsh chemical treatments, enabling the construction of living biofilm-derived materials in a large-scale and environmentally-stable manner.

### Definitions

It is to be understood that the figures and descriptions of the embodiments herein have been simplified to illustrate elements that are relevant for clear understanding, while eliminating, for the purpose of clarity, many other elements typically found in the art. Those of ordinary skill in the art may recognize that other elements and/or steps are desirable and/or required in implementing the present embodiments. However, because such elements and steps are well known in the art, and because they do not facilitate a better understanding of the present embodiments, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which these embodiments belong. Although any methods and materials similar or equivalent to those described herein can be used in the practice or testing of the present embodiments, the exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the embodiments can be presented in a range format. It should be understood that the description in range format is merely for convenience and brevity and should not be construed as an inflexible limitation on the scope of the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### 3D Printed Biofilm

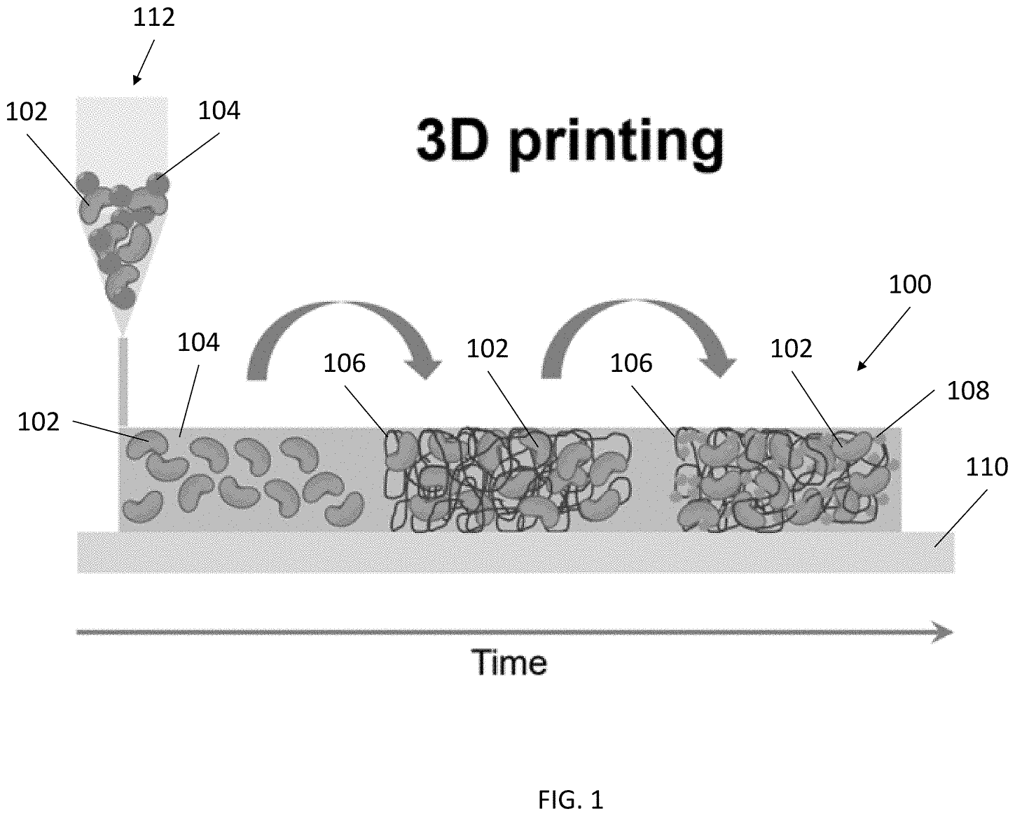

Referring now to FIG. 1, an exemplary biofilm 100 is depicted. Biofilm 100 comprises at least one population of microorganism 102 embedded within a matrix 104. The at least one population of microorganism 102 can include any number or combination of microorganisms selected from bacteria, viruses, protozoa, amoeba, algea, fungi, and the like. In some embodiments, the at least one population of microorganism 102 includes Escherichia coli, Bacillus subtilis, Bacteroides fragilis, Bifidobacterium bifidum, Enterobacter cloacae, Enterococcus faecalis, Methanobrevibacter smithii, Neisseria meningitides, Neisseria sicca, Staphylococcus aureus, Staphylococcus epidermidis, Streptococcus mutans, Streptococcus sanguinis, Streptococcus gordonii, Streptococcus salivarius, Actinomyces naeslundii, and combinations thereof. Matrix 104 can comprise any suitable material, including but not limited to gelatin, agarose, hyaluronic acid, fumed silica, κ-carrageenan, cellulose, collagen, fibrin, fibrinogen, thrombin, elastin, laminin, fibronectin, vitronectin, chondroitin 4-sulfate, chondroitin 6-sulfate, dermatan sulfate, heparin sulfate, vixapatin (VP12), heparin, keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. In certain embodiments, matrix 104 can further include one or more carbohydrates such as chitin, chitosan, alginic acids, and alginates such as calcium alginate and sodium alginate. These materials may be isolated from plant products, humans, or other organisms or cells or synthetically manufactured.

In various embodiments, biofilm 100 further comprises a plurality of matrix molecules, including but not limited to proteins 106, peptides 108, enzymes, amino acids, nucleic acids, vitamins, hormones, antibodies, growth factors, nanoparticles, microparticles, liposomes, viral and non-viral transfection systems, and the like. Additional contemplated additives include but are not limited to sugars, such as sucrose, fructose, cellulose, or mannitol; nutrients, such as bovine serum albumin; vitamins, such as vitamin B2, vitamin Ad, Vitamin D, Vitamin E, and Vitamin K; natural or synthetic steroids and hormones, such as dexamethasone, hydrocortisone, estrogens, and its derivatives; growth factors, such as fibroblast growth factor (FGF), transforming growth factor beta (TGF-β), and epidermal growth factor (EGF); and delivery vehicles, such as nanoparticles, microparticles, liposomes, viral and non-viral transfection systems. In some embodiments, the matrix molecules are added to the matrix during or after fabrication as an additive. In some embodiments, the at least one population of microorganism 102 produces the matrix molecules that permeate matrix 104.

In some embodiments, biofilm 100 further comprises one or more populations of native or genetically modified host cells, such that the populations of microorganisms 102 are co-cultured with the populations of host cells. In certain embodiments, the host cells are mammalian cells, e.g., human cells, which may interact with microorganisms 102 or biofilm 100, such as to generate matrix molecules. Non-limiting examples of contemplated cells include pluripotent stem cells, embryonic stems cells, hematopoietic stem cells, adipose derived stem cells, bone marrow derived stem cells, fibroblasts, osteocytes, epithelial cells, cardiomyocytes, endothelial cells, neurocytes, and combinations thereof. Suitable cells can also include cancer cells, including but not limited to: leukemia, lymphoma, myeloma, breast cancer, prostate cancer, endometrial cancer, bladder cancer, brain cancer, cervical cancer, lung cancer, melanoma, cervical cancer, ovarian cancer, colorectal cancer, pancreatic cancer, esophageal cancer, kidney cancer, thyroid cancer, liver cancer, uterine cancer, soft tissue sarcoma, bone cancer, stomach cancer, and combinations thereof.

Isolated cells may be cultured in vitro to increase the number of cells available for addition to biofilm 100 or bioink 112. The use of autologous cells can reduce or prevent tissue rejection typically seen with allogeneic cells. However, if an immunological response does occur in the subject after implantation of the artificial organ, the subject may be treated with immunosuppressive agents such as cyclosporin or FK506 to reduce the likelihood of rejection. In certain embodiments, chimeric cells, or cells from a transgenic animal, may be used.

Isolated cells may be transfected prior to coating with genetic material. Useful genetic material may be, for example, genetic sequences which are capable of reducing or eliminating an immune response in the host. For example, the expression of cell surface antigens such as class I and class II histocompatibility antigens may be suppressed. This may allow the transplanted cells to have reduced chances of rejection by the host. In addition, transfection could also be used for gene delivery.

Seeded cells may be normal or genetically engineered to provide additional or normal function. Methods for genetically engineering cells with retroviral vectors, polyethylene glycol, or other methods known to those skilled in the art may be used. These include using expression vectors which transport and express nucleic acid molecules in the cells. (See Goeddel; Gene Expression Technology: Methods in Enzymology 185, Academic Press, San Diego, Calif. (1990). Vector DNA may be introduced into prokaryotic or cells via conventional transformation or transfection techniques. Suitable methods for transforming or transfecting host cells can be found in Sambrook et al. (Molecular Cloning: A Laboratory Manual, 3nd Edition, Cold Spring Harbor Laboratory press (2001)), and other laboratory textbooks.

In various embodiments, biofilm 100 further comprises one or more therapeutics. The therapeutics can be natural or synthetic drugs, including but not limited to: analgesics, anesthetics, antifungals, antibiotics, anti-inflammatories, nonsteroidal anti-inflammatory drugs (NSAIDs), anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents.

In various embodiments, biofilm 100 can be fabricated by depositing a bioink 112 on a substrate 110. Bioink 112 can include the at least one population of microorganisms 102 and matrix 104. In some embodiments, bioink 112 further includes one or more matrix molecules including proteins 106 and peptides 108. In certain embodiments, bioink 112 includes one or more stabilizers, diluents, dispersing agents, suspending agents, thickening agents, and/or excipients. Substrate 110 can be any suitable substrate, including but not limited to metals, glass, wood, fabrics, fibers, polymers, and plastics, as well as any of the matrix materials described elsewhere herein. In some embodiments, biofilm 100 further comprises one or more polymerizers or crosslinkers that enhance gelation or solidification of bioink 112. The one or more polymerizers or crosslinkers can be applied prior to the deposition of bioink 112 such as a surface treatment on substrate 110, at the same time as bioink 112 such as with an adjacent nozzle or applicator, or after the deposition of bioink 112 on substrate 110. In some embodiments, the surface treatment includes calcium.

Deposition can be achieved using any technique, including but not limited to inkjet printing, extrusion, screen printing, electrospinning, spin coating, sputtering, rolling, spraying, and the like. Fabrication of biofilm-derived functional materials has been further developed with the aid of 3D printing technology. Commercial do-it-yourself 3D printers or construction toys have been recently demonstrated to be capable of printing bacteria via straight-forward alginate chemistry (Lehner B A E et al., ACS Synth Biol, 2017, 6:1124-1130; Schmieden D T et al., ACS Synth Biol, 2018, 7:1328-1337). These represent simple, scalable, and inexpensive approaches for printing biofilms with sub-millimeter precision that can mimic the spatial heterogeneity of natural biofilms. The spatial resolution of the 3D-printed biofilms is determined by multiple factors including the bioink composition, the concentration of chemicals that induce expression of the modified biofilm proteins, the rheological properties of the bioink, the biocompatibility of the ink with the printed bacteria, the surface smoothness of the printing substrate, and the like. 3D printing of bacteria has also been successfully achieved using bioink compositions including gelatin, agarose, hyaluronic acid, fumed silica, and κ-carrageenan (Schaffner M et al., Sci Adv, 2017, 3:eaao6804; Gonzalez L M et al., bioRxiv, 2019, 537571).

Previously, one major challenge of 3D bioprinting technology was the operating cost. This problem has been addressed by keeping the cost of customized 3D bioprinters to approximately 350 US dollars (Lehner B A E et al., ACS Synth Biol, 2017, 6:1124-1130; Schmieden D T et al., ACS Synth Biol, 2018, 7:1328-1337). Additionally, some inexpensive commercially available 3D printers can perform multi-channel printing, which can mix several input components and can to be repurposed to print bacteria. As a first example, it has been recently shown that 3D printing of bacterial spores with good resolution can be achieved with a customized multi-channel printing system, operating at higher temperatures (Gonzalez L M et al., bioRxiv, 2019, 537571). While this printer costs several times more than the low-cost customized 3D bioprinters, its multi-channel printing capability provides the option to keep the bacterial cells separated from the bioink scaffold components under different optimal conditions until printing. Creation or repurposing of cost-effective 3D printers that can perform multi-channel printing without heating the samples is ideal for 3D printing of bacteria and engineered biofilms with extended usage applications.

Given the wide repertoire of natural and artificial biopolymers, diverse synthetic biofilms can be 3D-printed for the creation of bacterially-inspired materials with tunable multi-scale patterning (Chen A Y et al., Nat Mater, 2014, 13:515-523; Nguyen P Q, Biochem Soc Trans, 2017, 45:585-597; Chen A Y et al., ACS Synth Biol, 2015, 4:8-11). For instance, bacteria in 3D-printed synthetic biofilms can aid in the production of biopolymers such as cellulose, curdlan, and other materials with improved mechanical or electrically conductive properties with biomedical and biotechnological applications (Schaffner M et al., Sci Adv, 2017, 3:eaao6804).

In some embodiments, biofilm 100 can be provided with substrate 110 attached. In other embodiments, biofilm 100 can be provided without substrate 110. In various embodiments, a plurality of biofilm 100, each having the same or different populations of microorganisms 102 and matrix 104, can be combined together to form a biofilm construct, such as by layering, stacking, encapsulating, molding, or by forming complex three dimensional shapes and constructs. Individual biofilms 100 and the biofilm construct can each have any desired size, shape, geometry, and patterning.

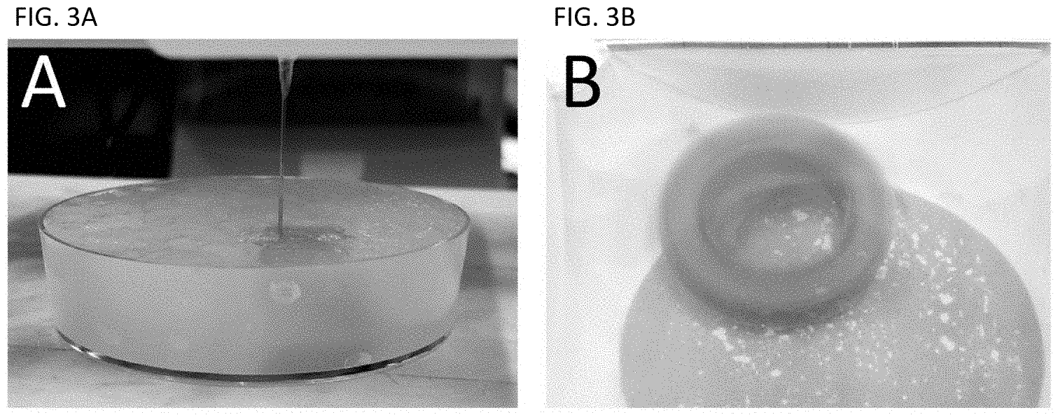

Referring now to FIG. 3A and FIG. 3B, a free-form printing method is now described. The free-form printing method enables mold-free manufacturing of arbitrary shapes and allows for the generation of tall, thin shapes or shapes with internal cavities that would otherwise collapse during standard deposition. To achieve this, the method implements a sacrificial suspension media. For example, FIG. 3A depicts the use of an agarose slurry as a suspension media. However, contemplated suspension media are not limited to agarose and can include gels, pastes, particulate composition, and the like. The suspension media behaves as a non-Newtonian fluid that holds a printed material in place while allowing a printhead or nozzle to move through the suspension media to deposit the printed material. Immersion within the suspension media allows soft bioinks to be stabilized (FIG. 3B) during bioink polymerization or biofilm matrix production to form arbitrary structures with extreme aspect ratios. Once biofilm printing is complete, the suspension media may be removed, or the biofilm may simple be lifted out of the suspension media. In some embodiments, the suspension media can contain polymerizing or crosslinking agents. For example, the suspension media can contain Ca²⁺ in the case of alginate-based bioinks to promote rapid polymerization and to improve printing resolution. The ability to create internal cavities also allows the generation of lattice structures. In some embodiments, the printing method can be augmented using computer-aided design (CAD) structural designing using topology optimization techniques that computationally identify optimized structural forms to carry specified loading to support locations.

### Printing Apparatus

The present disclosure also includes printing apparatuses. As described elsewhere herein, biofilms are printed from extruded bioinks that include at least two components, a matrix and microorganisms and other molecules. In some embodiments, the bioinks are premixed prior to printing. In some embodiments, bioinks may be provided as separate components that are combined just prior to printing. In some embodiments, bioinks may be printed in combination with other compositions that are combined just prior to printing. In various embodiments, it is advantageous to keep ink components separated until printing, such as to prolong stability or viability. Thus, various printing apparatuses are provided to enhance mixing and combination of ink compositions at the time of printing.

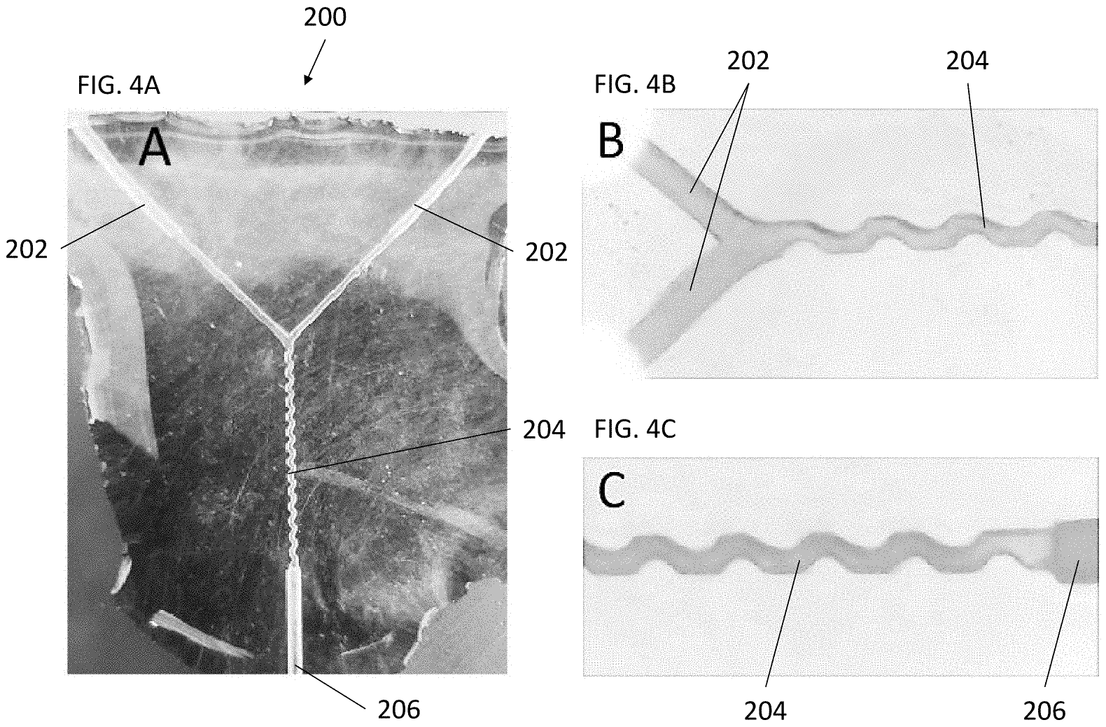

Mixing of input bioinks in a microfluidic setting poses an inherent challenge due to low Reynolds numbers from extremely small hydraulic diameters, leading to laminar flows and low turbulence. To overcome this difficulty, microfluidic structures that increase turbulence are provided for passive mixing. Referring now to FIG. 4A through FIG. 4C, an exemplary passive mixer 200 is depicted. Passive mixer 200 comprises at least two inlet channels 202, wherein each inlet channel 202 fluidly join together into a mixing channel 204 that is in turn fluidly connected to an outlet channel. Mixing channel 204 comprises one or more physical structures that increase turbulence to promote passive mixing. In some embodiments, the physical structures can include undulations (as shown in FIG. 4A through FIG. 4C in the form of semicircular bumps), twists, turns, or any other variations in pathing of mixing channel 204. In some embodiments, the physical structures can include pegs, nodules, fins, or any other physical structures embedded in a fluid path of mixing channel 204 (not pictured). The elements of passive mixer 200 can be embedded in a solid block or provided as freestanding lumens.

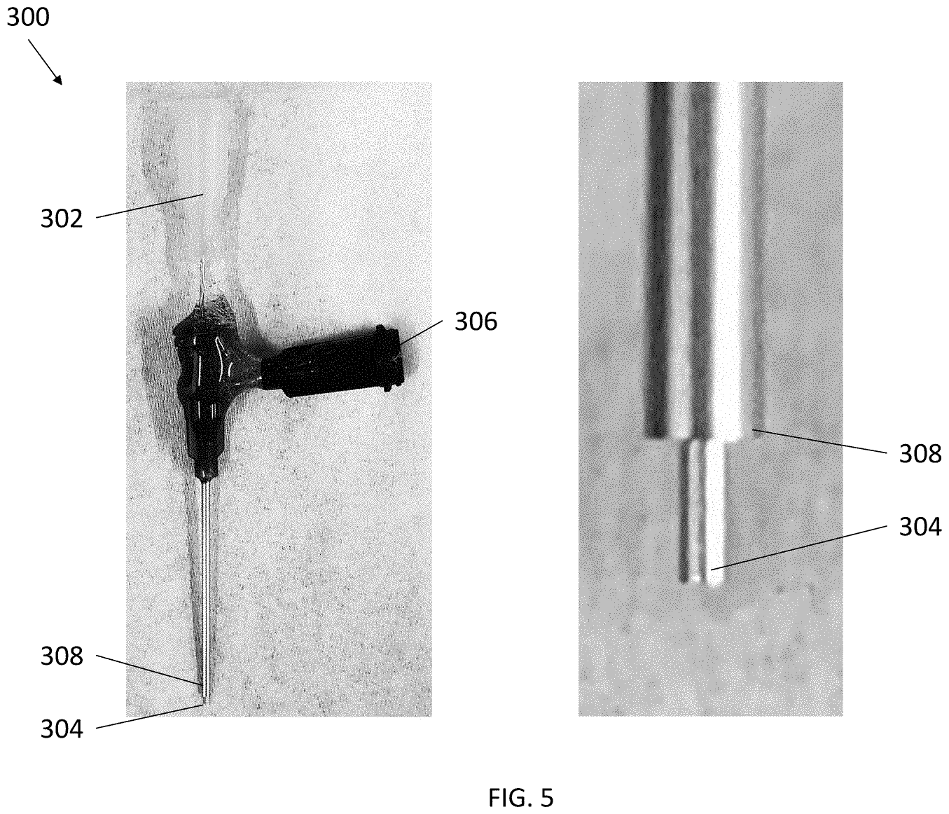

In some embodiments, the present disclosure provides multi-input printing nozzles, wherein the nozzles simultaneously deposit two or more matrices. In this manner, matrices can be combined or mixed at the point of deposition. In some embodiments, matrices that rely on a substrate for polymerization or crosslinking can be extruded with a polymerizing agent or crosslinking agent independent of a substrate of a particular type or composition. The matrices can also be deposited in a controlled layer-by-layer pattern, such as to mimic the natural heterogeneity of natural biofilms. Referring now to FIG. 5, an exemplary multi-input printing nozzle 300 is depicted. Printing nozzle 300 comprises at least a first nozzle 302 having a first nozzle tip 304 and a second nozzle 306 having a second nozzle tip 308. It should be understood that printing nozzle 300 is not limited to a first nozzle 302 and a second nozzle 306, and can include any number of nozzles. Printing nozzle 300 extrudes matrices such that the extruded matrices immediately and physically contact each other as they exit the nozzle tip. In some embodiments, the nozzles are nested within each other, such that smaller diameter nozzle tips are positioned within larger diameter nozzle tips. For example, FIG. 5 depicts first nozzle tip 304 nested within second nozzle tip 306. In some embodiments, the nozzles are positioned adjacent to each other. In some embodiments, each nozzle tip terminates at an equal position of printing nozzle 300. In some embodiments, such as in FIG. 5, each nozzle tip is staggered, wherein smaller diameter nozzle tips extend farther than larger diameter nozzle tips.

### Methods of Use

The present disclosure also includes methods of using biofilms. As described elsewhere herein, the present disclosure combines microorganisms with a matrix material to form biofilms. The microorganisms can be genetically modified and the matrix materials can be tuned to alter the physical attributes of a biofilm for numerous applications.

3D-printed biofilms functionalized with synthetic enzymes can aid in the processing of materials even under conditions of adverse pH, temperature, or exposure to organic solvents. The desired biocatalytic transformation occurs due to the enzymes that are irreversibly immobilized in the extracellular matrix of these biofilms. The enhanced mass transfer rates and surface area in these biofilms results in increased enzymatic activities. Such biofilms can be engineered to produce scaffolded chemical pathways, in which successive chemical reactions are catalyzed by individual stacked layers of the same or different bacteria (differing in species, strains, or genetic makeup), leading to production of a single product or a series of products via a relay of reactions. As one example, the printed bacteria is genetically manipulated to perform complex logic gate functions (Tamsir A et al., Nature, 2011, 469:212-215), such that the output of one layer serves as the input to the adjacent layer (Osmekhina E et al., Commun Biol, 2018, 1:97). These sequential reactions proceed more efficiently in 3D-printed biofilms due to the free diffusion of molecules between the stacked layers and their minimal separation distance, thus leading to multi-step transformations. Alternatively, templated assembly of nanoparticles on engineered biofilms can be used to catalyze multi-step hybrid reaction systems (Nguyen P Q et al., Nat Commun, 2014, 5:4945; Huang J et al., Nat Chem Biol, 2019, 15:34-41).

Non-engineered or engineered beneficial bacterial biofilms can be 3D-printed as an anti-fouling coating on building or marine vessel surfaces. These living functional bacteria use up the oxygen on the surface and in turn produce compounds that are anti-corrosive, thereby preventing corrosion and biofouling. Similarly, probiotic biofilms can be 3D-printed onto various biomedical implant surfaces to prevent device-associated infections caused by pathogenic bacteria.

3D-printed engineered biofilms can be deployed for environmental detoxification purposes including bioremediation, abstraction of rare earth elements (REEs) and heavy metals, removal of assimilable organic carbon, and in wastewater treatment plants (Schaffner M et al., Sci Adv, 2017, 3:eaao6804; Tay P K R et al., Green Chemistry, 2018, 20:3512). Bringing together the higher metabolic potential and specific catabolic nature of active bacteria with the increased surface area and chemical resilience of the biofilm matrix enables patterned, engineered biofilms to act as a sink capable of absorption and degradation of chemicals from processing liquid streams. Synthetic biofilms displaying selected catabolic enzymes, heavy metal binding proteins, inorganic nanoparticles, or REE-binding domains can be 3D-printed onto filters or onto pipes and reactors in treatment plants to carry out the desired degradation or abstraction activities as the contaminating streams flow past. Analytical techniques such as HPLC-MS or ICP-MS can be used to quantify the amount of chemicals absorbed onto the biofilm matrix components, wherein the bound residues are desorbed with simple acidic or alkaline washes. Metal-binding domains can be additionally added to these synthetic biofilms to facilitate their strong surface attachment such that they resist detachment forces and withstand multiple sorption-desorption cycles. With appropriate tuning of the bioink porosity, such 3D-printed biofilms are recyclable and reusable with minimum loss of efficiency. Incorporation of feedback-regulated genetic circuits can be used in situations involving continuous detoxification such that synthetic biofilms are produced only when the specific target chemical is sensed, thereby improving the overall absorption efficiencies.

3D printing can be employed to investigate interactions between bacteria species in mixed biofilms or between bacterial biofilms with their eukaryotic hosts. These experiments can be performed by (a) incorporating different bacteria in the same bioink, (b) printing different bacterial bioinks adjacent to each other with shared interfaces, and/or (c) printing layers of host cells overtop of existing mature 3D-printed biofilms or vice versa. Following appropriate exposure times, imaging techniques and -omics approaches (transcriptomics, proteomics, or metabolomics) can then be used on both the bacterial and host samples to decipher their communication and community behavior.

In natural biofilms, factors like the density of the bacteria and the extracellular matrix components, the distribution of nutrients and signaling molecules, the locations of water channels, and the distribution of molecular oxygen are dynamic variables. The consequences of these variables on the emergent biological (metabolic heterogeneity and antibiotic resistance) and mechanical (cohesiveness, viscoelasticity, resistance to hydrodynamic shear and desiccation) phenotypes in biofilms are not well characterized. 3D printing can be informative in this regard to identify the design principles of biofilms by introducing individual variations in the 3D spatial distribution of biofilm constituents and studying their resultant attributes of biological and mechanical endurance. 3D printed biofilm systems can also mimics the robustness of natural biofilms whilst maintaining their structure-function relationships over time. Such biofilm systems are useful for practical applications such as testing potential anti-biofilm treatments, evaluating the adequacy of mathematical models of biofilms, and the like.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Resistance to Anti-Bacterial Treatment

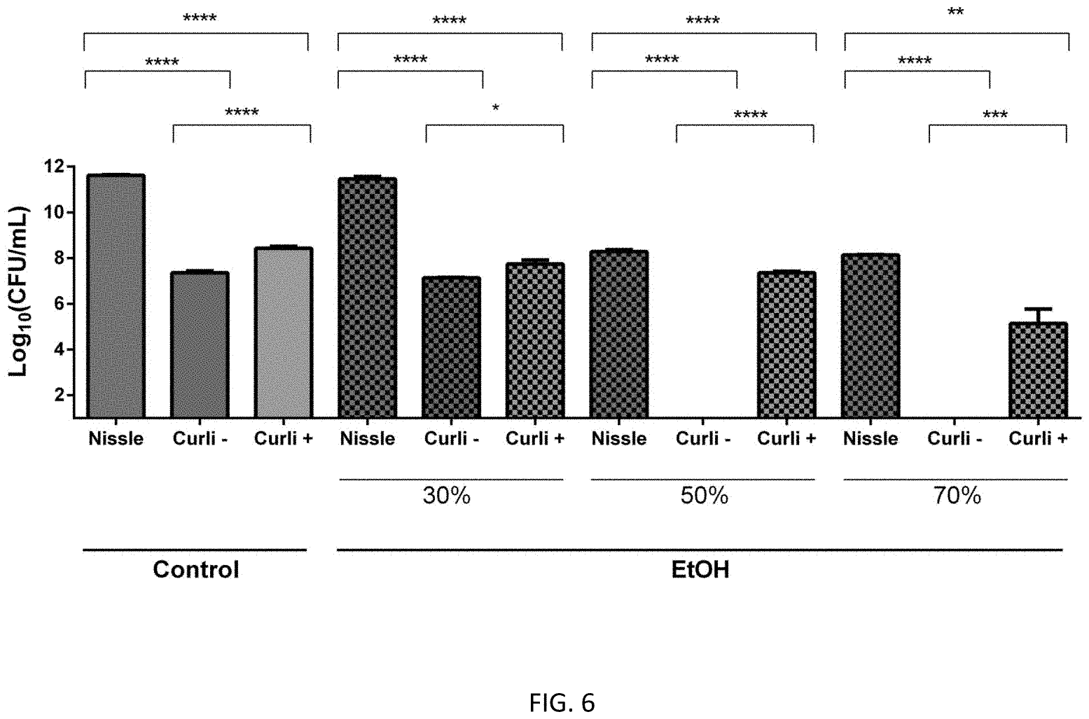

Natural biofilms develop robust resistance to many anti-bacterial treatments. Here, four layers of bio-ink were 3D-printed, containing either E. coli Nissle (a natural biofilm-forming strain), K-12 E. coli (curli− non-biofilm forming lab strain), or K-12 E. coli engineered to express curli fibers (curli+). After incubation for 5 days to form a biofilm, a 1 cm² sample was cut out and treated with varying concentrations of ethanol, a chemical with anti-bacterial activity. The colony forming units was then determined to quantify the number of bacteria that had survived the ethanol treatment. The results showed that the engineered biofilms survived the ethanol much better than the non-engineered bacteria, and nearly as well as the natural biofilm-forming strain (FIG. 6). Therefore, it was shown that engineered 3D-printed biofilms develop resistance to anti-bacterial treatments, similar to natural biofilms.

### Example 2: Oxygen Depletion within Biofilm

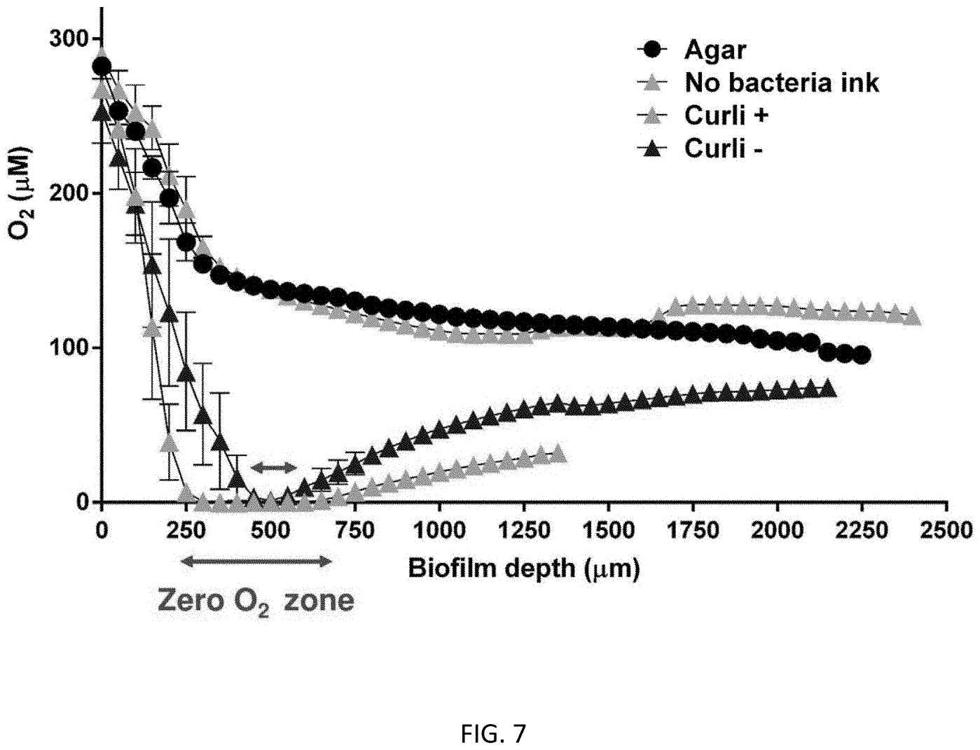

Natural biofilms deplete the oxygen within themselves, creating a deep zone of low or zero oxygen. Here, four layers of bio-ink were 3D-printed, containing either no bacteria, K-12 E. coli (curli− non-biofilm forming lab strain), or K-12 E. coli engineered to express curli fibers (curli+). After incubation for 7 days to form a biofilm, a probe was used to determine the concentration of soluble oxygen at different depths within the printed structures. The results showed that the engineered biofilms had a zero-oxygen zone that spanned more than 250 um, while the non-engineered bacteria had a very shallow zero-oxygen zone of approximately 25 um in depth and the bio-ink containing no bacteria did not have any regions of zero oxygen (FIG. 7). Therefore, it was shown that the 3D-printed biofilms developed a deep zone of zero oxygen, similar to natural biofilms.

### Example 3: Development of Microphysiological Platform

The following study exploits 3D bioprinting technology described elsewhere herein to reproducibly create biofilms that simulate biological diversity within the oral cavity on a chip platform for use in high-throughput drug and drug-delivery vehicle screening. Model oral biofilms are developed and applied to prevent growth of cariogenic bacteria.

Multispecies Quiescent and Cariogenic Biofilm-On-a-Chip Using 3D Printing

A combination of commensals, including S. sanguinis, S. gordonii, S. salivarius, and A. naeslundii are 3D-printed to form quiescent plaque on tooth-mimicking commercial hydroxyapatite surfaces. Cariogenic biofilms are also developed by printing S. mutans in the majority along with Lactobacillus and Bifidobacterium species, consistent with cariogenic biofilms.

The high spatial control of the bacterial deposition process is applied to reproduce the complex three-dimensional patterning of native biofilm components. For example, native biofilms have higher concentrations of bacteria in lower regions, and higher concentrations of EPS substances in upper regions. Successful biofilm emulation is verified via resistance to antibacterial treatments using colony-forming unit or live/dead assays; measurement of gradients of oxygen depletion (using a dissolved oxygen probe) and metabolic activity (via fluorescence microscopy detection or aerobic respiration markers); and ability to cause increased pH and demineralize dentin-mimetic hydroxyapatite discs.

These measurements are analyzed versus metrics determined for native quiescent and cariogenic biofilms. These results provide feedback for adjusting 3D-printing parameters, including spatial organization and ratios of bacterial/fungal species, and to attain robust biological biofilm properties. Recapitulation of quiescent and cariogenic biofilms is achieved when chip conditions are found resulting in <0.5-log reduction in viability with 70% ethanol treatment, free O₂<5 μM for >50% of biofilm depth, and pH<5.5 (for cariogenic biofilms).

Quiescent Biofilm Prevents Cariogenic S. mutans Colonization and Growth

Bioprinted quiescent dental plaque is tested for its ability to prevent colonization and growth of caries-causing S. mutans bacteria in the mouth. Models of quiescent oral biofilms are 3D-printed onto tooth-mimicking hydroxyapatite discs (to mimic dentin and dental implant materials) and incubate them with fluorescently labeled S. mutans bacteria. Proliferation and invasion of S. mutans is assayed visually by fluorescence microscopy and quantitatively by fluorescent cell sorting and qPCR techniques, in comparison to controls with no quiescent oral biofilm present. Success criteria is ≥2 log reduction in adherent S. mutans bacteria.

The disclosures of each and every patent, patent application, and publication cited herein are hereby incorporated herein by reference in their entirety. While this disclosure has been disclosed with reference to specific embodiments, it is apparent that other embodiments and variations may be devised by others skilled in the art without departing from the true spirit and scope of the disclosure. The appended claims are intended to be construed to include all such embodiments and equivalent variations.

What is claimed is:

1. A biofilm, comprising:
a layer of matrix material; and
at least one population of microorganisms positioned within the layer of matrix material;
wherein the at least one population of microorganisms produce at least one molecule that permeates the matrix material.

2. The biofilm of claim 1, wherein the at least one population of microorganisms is selected from the group consisting of: bacteria, viruses, protozoa, amoeba, algae, and fungi.

3. The biofilm of claim 2, wherein the at least one population of microorganisms is genetically altered.

4. The biofilm of claim 2, wherein the at least one population of microorganisms is selected from the group consisting of: Escherichia coli, Bacillus subtilis, Bacteroides fragilis, Bifidobacterium bifidum, Enterobacter cloacae, Enterococcus faecalis, Methanobrevibacter smithii, Neisseria meningitides, Neisseria sicca, Staphylococcus aureus, Staphylococcus epidermidis, Streptococcus mutans, Streptococcus sanguinis, Streptococcus gordonii, Streptococcus salivarius, and Actinomyces naeslundii.

5. The biofilm of claim 1, wherein the layer of matrix material further comprises one or more populations of host cells.

6. The biofilm of claim 1, wherein the layer of matrix material is selected from the group consisting of: gelatin, agarose, hyaluronic acid, fumed silica, κ-carrageenan, cellulose, collagen, fibrin, fibrinogen, thrombin, elastin, laminin, fibronectin, vitronectin, chondroitin 4-sulfate, chondroitin 6-sulfate, dermatan sulfate, heparin sulfate, vixapatin (VP12), heparin, and keratan sulfate, proteoglycans, chitin, chitosan, alginic acids, alginates, and combinations thereof.

7. The biofilm of claim 6, wherein the layer of matrix material further comprises one or more matrix molecule selected from the group consisting of: proteins, peptides, enzymes, amino acids, nucleic acids, vitamins, hormones, antibodies, growth factors, nanoparticles, microparticles, liposomes, viral and non-viral transfection systems, therapeutics, and drugs.

8. The biofilm of claim 1, wherein the biofilm is fabricated by the deposition of a bioink on a substrate, the bioink comprising the at least one population of microorganisms and a matrix material.

9. The biofilm of claim 8, wherein the substrate is selected from the group consisting of: gelatin, agarose, hyaluronic acid, fumed silica, κ-carrageenan, cellulose, collagen, fibrin, fibrinogen, thrombin, elastin, laminin, fibronectin, vitronectin, chondroitin 4-sulfate, chondroitin 6-sulfate, dermatan sulfate, heparin sulfate, vixapatin (VP12), heparin, and keratan sulfate, proteoglycans, chitin, chitosan, alginic acids, alginates, metals, glass, wood, fabrics, fibers, polymers, plastics, and combinations thereof.

10. The biofilm of claim 8, wherein the fabrication of the biofilm further includes the application of a polymerizer or a crosslinker.

11. The biofilm of claim 10, wherein the polymerizer or crosslinker comprises calcium.

12. The biofilm of claim 8, wherein the deposition is a method selected from the group consisting of: 3D printing, inkjet printing, extrusion, screen printing, electrospinning, spin coating, sputtering, rolling, and spraying.

13. The biofilm of claim 1, wherein the biofilm comprises enzymes suitable for processing materials under extreme pH, temperature, and solvent conditions.

14. The biofilm of claim 1, wherein the biofilm is an anti-fouling coating having bacteria that produce anti-corrosive compounds.

15. The biofilm of claim 1, wherein the biofilm is a detoxifying matrix having catabolic enzymes, heavy metal binding proteins, inorganic nanoparticles, rare earth element (REE) binding domains, and combinations thereof.

16. The biofilm of claim 15, wherein the biofilm is recyclable and reusable.

17. The biofilm of claim 1, further comprising one or more additional layers of matrix material.

18. The biofilm of claim 17, wherein at least one of the additional layers of matrix material comprises at least one population of microorganisms.

19. The biofilm of claim 18, wherein the additional layers of matrix material each comprise different populations of microorganisms.

20. A method of fabricating a biofilm, comprising the steps of:
providing at least one bioink, each bioink comprising at least one population of microorganisms suspended in a matrix material; and
depositing the at least one bioink onto a substrate;
wherein the at least one population of microorganisms produce at least one molecule that permeates the matrix material.

21. The method of claim 20, wherein the substrate is a suspension media comprising a non-Newtonian fluid, such that the at least one bioink is depositable in three-dimensional space within the suspension media.

22. The method of claim 20, wherein the at least one bioink is extruded through a passive mixer with at least one additional composition, wherein the passive mixer comprises at least two inlet channels fluidly joining together into a mixing channel that is fluidly connected to an outlet channel.

23. The method of claim 22, wherein the mixing channel comprises at least one turbulence-increasing physical structure.

24. The method of claim 23, wherein the physical structure is selected from the group consisting of: channel path undulations, embedded pegs, embedded nodules, and embedded fins.

25. The method of claim 20, wherein the at least one bioink is extruded through a multi-input printing nozzle with at least one additional composition, wherein the multi-input nozzle comprises at least one first nozzle having a first nozzle tip and at least one second having a second nozzle tip.

26. The method of claim 25, wherein the first nozzle tip has a diameter that is smaller than a diameter of the second nozzle tip.

27. The method of claim 26, wherein the first nozzle tip is nested within the diameter of the second nozzle tip.

28. The method of claim 25, wherein the first nozzle tip is positioned adjacent to the second nozzle tip.

29. The method of claim 25, wherein the first nozzle tip and the second nozzle tip terminate at an equal position relative to the multi-input printing nozzle.

30. The method of claim 25, wherein the first nozzle tip and the second nozzle tip terminate at different positions relative to the multi-input printing nozzle.
